(19) United States
(12) Patent Application Publication
(10) Pub. No.: US 2010/0227809 A1
(43) Pub. Date: Sep. 9, 2010

(54) COMBINATION TREATMENT FOR METABOLIC DISORDERS

(75) Inventors: Stephen D. Wolpe, Boyds, MD (US); Ramachandran Arudchandran, Germantown, MD (US); Reid W. von Borstel, Potomac, MD (US)

Correspondence Address: Wellstat Management Company. LLC, LEGAL DEPARTMENT, 930 CLOPPER ROAD, GAITHERSBURG, MD, 20878, US

(73) Assignee: WELLSTAT THERAPEUTICS CORPORATION, Gaithersburg, MD (US)

(21) Appl. No.: 12/377,460

(22) PCT Filed: Aug. 16, 2007
(86) PCT No.: PCT/US07/76116

§ 371 Date: Feb. 13, 2009

Related U.S. Application Data
(60) Provisional application No. 60/822,676, filed on Aug. 17, 2006.

(51) Int. Cl. A61K 38/17; A61P 3/00

(52) U.S. Cl. 514/12

(57) ABSTRACT

Various metabolic disorders, such as insulin resistance syndrome, diabetes, polycystic ovary syndrome, hyperlipidemia, fatty liver disease, cachexia, obesity, atherosclerosis and arteriosclerosis can be treated with a compound selected from an incretin mimetic and a dipeptidyl peptidase IV inhibitor in combination with a Compound of Formula, I or a pharmaceutically acceptable salt thereof, Formula (I) Three of R¹, R², R³, R⁴ and R⁵ are hydrogen and the remainder are independently selected from the group consisting of hydrogen, halo, hydroxy, methyl, ethyl, perfluoromethyl, methoxy, ethoxy, and perfluoromethoxy; and m is 0, 2 or 4. R⁶ is hydrogen, O or hydroxy, and X is —OR⁷, wherein R⁷ is hydrogen or alkyl having from 1 to 3 carbon atoms; or R⁶ is hydrogen, and X is —NR⁸R⁹, wherein R⁸ is hydrogen or hydroxy and R⁹ is hydrogen, methyl or ethyl. When X is —NR⁸R⁹, hydroxy none of R¹, R², R³, R⁴ and R⁵ is hydroxy.

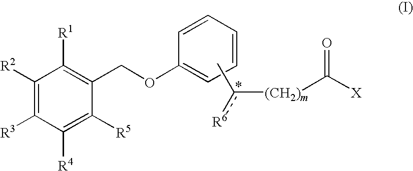

### BACKGROUND OF THE INVENTION

Diabetes is a major and growing public health problem. Late stage complications of diabetes consume a large proportion of national health care resources.

The use of the Compounds of Formula I in combination with certain other drugs to treat diabetes or insulin resistance syndrome is disclosed in WO 02/100341, WO/073611, WO 04/091486, WO 2006/127133 and International Patent Application No. PCT/US07/60833, all of which are assigned to Wellstat Therapeutics Corp.

Exendin-4 has been tested in combination with metformin, with an antidiabetic sulfonylurea, and with a thiazolidinedione.

### SUMMARY OF THE INVENTION

This invention concerns therapeutic uses of a compound selected from the group consisting of an incretin mimetic and a dipeptidyl peptidase IV (DPPIV) inhibitor, in combination with a Compound of Formula I or a pharmaceutically acceptable salt thereof.

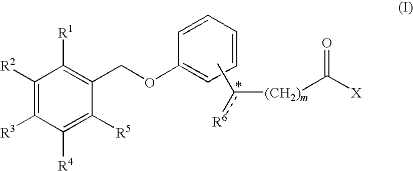

In Formula I, m is 0, 2 or 4. X is —OR⁷, wherein R⁷ is hydrogen or alkyl having from 1 to 3 carbon atoms; R⁶ is hydrogen, O or hydroxy; and three of R¹, R², R³, R⁴ and R⁵ are hydrogen and the remainder are independently selected from the group consisting of hydrogen, halo, hydroxy, methyl, ethyl, perfluoromethyl, methoxy, ethoxy, and perfluoromethoxy. Alternatively X is —NR⁸R⁹, wherein R⁸ is hydrogen or hydroxy and R⁹ is hydrogen, methyl or ethyl; R⁶ is hydrogen; and three of R¹, R², R³, R⁴ and R⁵ are hydrogen and the remainder are independently selected from the group consisting of hydrogen, halo, methyl, ethyl, perfluoromethyl, methoxy, ethoxy, and perfluoromethoxy.

This invention provides a method of treating a mammalian subject having a condition selected from the group consisting of insulin resistance syndrome, diabetes (both Type I diabetes and Type II diabetes), polycystic ovary syndrome, hyperlipidemia, fatty liver disease, cachexia, obesity, atherosclerosis and arteriosclerosis, comprising administering to the subject a Compound of Formula I or a pharmaceutically acceptable salt thereof and an incretin mimetic in a combined amount effective to treat the metabolic condition.

This invention provides the use of a biologically active agent in the manufacture of a medicament for treatment of a condition selected from the group consisting of insulin resistance syndrome, diabetes (both Type I Diabetes and Type II Diabetes), and polycystic ovary syndrome; or for the treatment or reduction in the chance of developing atherosclerosis, arteriosclerosis, obesity, hypertension, hyperlipidemia, fatty liver disease, nephropathy, neuropathy, retinopathy, foot ulceration or cataracts associated with diabetes; or for the treatment of a condition selected from the group consisting of hyperlipidemia, cachexia, and obesity; wherein the agent is a Compound of Formula I or a pharmaceutically acceptable salt thereof and is formulated for use in combination with an incretin mimetic in a combined amount effective to treat the metabolic condition.

A kit comprising one or more unit oral doses of a Compound of Formula I or a pharmaceutically acceptable salt thereof, one or more unit injectable doses of an incretin mimetic, and instructions for administering the Compound of Formula I or pharmaceutically acceptable salt thereof in combination with the incretin mimetic.

This invention is based on the finding that an incretin mimetic such as Exendin-4 amide, which is also a glucagon-like peptide-1 analog (GLP-analog) in combination with a compound of Formula I such as Compound BI provided superior antidiabetic activity than either compound alone, as shown in the Example. Both GLP-1 analogs and DPPIV inhibitors act largely through activation of GLP-1 receptors. GLP-1 analogs do so by direct binding of the receptor. DPPIV inhibitors do so by increasing endogenous levels of GLP-1. Therefore, the finding that Compound BI amplifies and enables antidiabetic activity of Exendin-4 amide supports combined use of Compound BI with the entire range of GLP-1 analogs and DPPIV inhibitors. Other antidiabetic analogs of Compound BI are also useful in this context.

Antidiabetic drugs based on GLP-1 (glucagon-like peptide-1) are emerging as clinically useful agents. GLP-1 itself is a peptide with a short half-life and is not suitable for use as a therapeutic agent. Longer lasting GLP-1 analogs have been discovered, devised and developed. Prominent among these is Exendin-4, a peptide derived from Gila-monsters (which eat only three times per year and use Exendin to generate a functional pancreas each time they eat) with homology to GLP-1 but a much longer half-life in vivo. Another emerging strategy involves inhibitors of the enzyme that breaks down GLP-1, dipeptidyl peptidase IV (DPPIV). GLP-1 analogs or modulators act primarily on pancreatic islets, increasing insulin output under conditions of hyperglycemia, and also reducing glucagon production. The net effect is to reduce blood glucose when it is elevated, but to a smaller degree when glucose is near normal.

### BRIEF DESCRIPTION OF THE FIGURES

FIG. 1: Serum glucose in db/db mice treated with Compound BI, exendin-4 amide, or both compounds.

FIG. 2: Islet insulin in db/db mice treated with Compound BI, exendin-4 amide, or both compounds.

FIG. 3: Serum insulin in db/db mice treated with Compound BI, exendin-4 amide, or both compounds.

FIG. 4: Serum glucose in db/db mice treated with Compound BI, exendin-amide, or both compounds.

FIG. 5: Pancreatic insulin in db/db mice treated with Compound BI, exendin-amide, or both compounds.

FIG. 6: Serum glucose in db/db mice treated with Compound BI, exendin-amide, or both compounds.

FIG. 7: Pancreatic insulin in db/db mice treated with Compound BI, exendin-amide, or both compounds.

FIG. 8: Serum glucose in streptozotocin-treated C57B1/6J mice treated with a combination of Compound BI and exendin-amide.

FIG. 9: Serum C-peptide in streptozotocin-treated C57B1/6J mice treated with a combination of Compound BI and exendin-amide.

FIG. 10: Pancreatic insulin in streptozotocin-treated C57B1/6J mice treated with a combination of Compound BI and exendin-amide.

FIG. 11: Serum glucose in db/db mice treated with P32/98, Compound BI, or both compounds.

FIG. 12: Pancreatic insulin in db/db mice treated with P32/98, Compound BI, or both compounds.

### DETAILED DESCRIPTION OF THE INVENTION

### Definitions

As used herein the term “alkyl” means a linear or branched-chain alkyl group. An alkyl group identified as having a certain number of carbon atoms means any alkyl group having the specified number of carbons. For example, an alkyl having three carbon atoms can be propyl or isopropyl; and alkyl having four carbon atoms can be n-butyl, 1-methylpropyl, 2-methylpropyl or t-butyl.

As used herein the term “halo” refers to one or more of fluoro, chloro, bromo, and iodo.

As used herein the term “perfluoro” as in perfluoromethyl or perfluoromethoxy, means that the group in question has fluorine atoms in place of all of the hydrogen atoms.

As used herein “Ac” refers to the group CH₃C(O)—.

The bond between R⁶ and the carbon atom to which it is directly bonded is depicted in Formula I above by a solid line together with a dashed line. This depiction reflects that the bond in question can be either a single bond, when R⁶ is hydrogen or hydroxy, or a double bond, when R⁶ is O.

The asterisk in the depiction of Formula I above indicates a possible chiral center, and that carbon is chiral when R⁶ is hydroxy. In such cases, this invention provides the racemate, the (R) enantiomer, and the (S) enantiomer, of the Compounds of Formula I, all of which are believed to be active. Mixtures of these enantiomers can be separated by using HPLC, for example as described in Chirality 11:420-425 (1999).

Certain chemical compounds are referred to herein by their chemical name or by the two-letter code shown below. Compounds BI, CF, CR and CT are included within the scope of Formula I shown above.

BI 4-(3-(2,6-Dimethylbenzyloxy)phenyl)-4-oxobutyric acid
CF 3-(2,6-Dimethylbenzyloxy)phenylacetic acid
CR 4-(3-(2,6-Dimethylbenzyloxy)-phenyl)-4(R)-hydroxybutanoic acid
CT N-Hydroxy-2-[3-(2,6-dimethylbenzyloxy)phenyl]acetamide

As used herein the term “incretin mimetic” means a compound that mimics the anti-diabetic actions of naturally occurring hormones called incretins.

“Exendins” are a class of peptides derived from Gila-monster venom, including truncated versions of such peptides. “Exendin agonists” are peptides and peptide mimetics based on the amino acid structure of exendins and having some or all of the activity of exendins. As used herein the term “exendin-4” encompasses both exendin-4 and exendin-4 amide, unless specifically excluded by the context. The terms “exendin-4 amide” and “exendin-amide” are used interchangeably.

Dipeptidyl peptidase IV (DPPIV) is an enzyme, also known as CD26, that breaks down GLP-1 (glucagon-like peptide-1). The term “dipeptidyl peptidase IV inhibitor” or “DPPIV inhibitor” means a compound that inhibits DPPIV activity.

As used herein the abbreviation “p.o.” means per os (orally). The abbreviation “ip” or “i.p.” means intraperitoneally.

As used herein the transitional term “comprising” is open-ended. A claim utilizing this term can contain elements in addition to those recited in such claim.

### Compounds of the Invention

In an embodiment of the invention described in the Summary above, R¹ is methyl and R⁵ is methyl. In another embodiment X is —OR⁷, wherein R⁷ is hydrogen or alkyl having from 1 to 3 carbon atoms. In another embodiment X is —NR⁸R⁹, wherein R⁸ is hydrogen or hydroxy and R⁹ is hydrogen, methyl or ethyl.

In a further embodiment of the invention described in the Summary above, the incretin mimetic or DPPIV inhibitor is combined with a compound of formula IA or a pharmaceutically acceptable salt thereof.

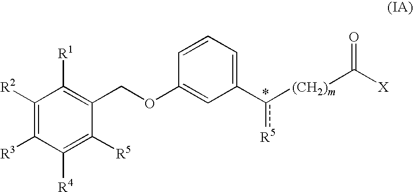

In Formula IA the variables have the same values as described above in connection with Formula I. Preferably R¹ is methyl and R⁵ is methyl. Examples of such Compounds include compounds BI, CF, CR and CT.

The compounds of Formula I can be made according to methods described in WO 02/100341, WO/073611, WO 04/091486, U.S. Provisional Patent Applications No. 60/667,457, filed Apr. 1, 2005, and No. 60/762,068, filed Jan. 25, 2006, the contents of which are incorporated herein by reference.

Examples of incretins include GLP-1. In accordance with the invention described above any incretin mimetic can be utilized. Examples of such incretin mimetics include exendins, exendin agonists, GLP-1 analogs, non-peptide small molecule GLP-1 receptor agonists, their polymer-modified and acylated forms and pharmaceutically acceptable salts, as well as hydrates and solvates of such compounds and such salts. Examples of such compounds can be found, inter alia, in U.S. Pat. Nos. 6,989,366, 6,506,724, and 6,924,264, and European Patent Publication No. EP01688148A1 all of which are incorporated herein by reference. Exendin-4 amide (exenatide; BYETTA) is currently marketed under the tradename BYETTA (Amylin Pharmaceuticals, Inc. and Eli Lilly and Co.). Examples of GLP-1 analogs include long-lasting GLP-1 analogs (e.g., liraglutide (Novo Nordisk), a GLP-1 acylated with a fatty acid chain), degradation-resistant GLP-1 analogs (e.g., GLP-1 analogs with amino acid substitutions to improve resistance to proteolytic degradation), GLP-1 analogs conjugated to serum proteins such as albumin (e.g., CJC-1131 and CJC-1134, Conjuchem), and peptides derived from GLP-1 that bind the GLP-1 receptor.

Exendin-4 is a 39-amino acid peptide having the following amino acid sequence: His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser. Exendin-4 amide is exendin-4 amidated by the addition of an —NH2 group at the C-terminus: His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH₂.

In accordance with this invention any DPPIV inhibitor can be utilized. Examples of such compounds include Vildagliptin (GALVUS, Novartis) (2S)-{[(3-hydroxyadamantan-1-yl)amino]acetyl}-pyrrolidine-2-carbonitrile; Sitagliptin (JANUVIA, Merck) (2R)-4-oxo-4-(3-[trifluoromethyl]-5,6-dihydro[1,2,4]triazolo[4,3-a]pyrazin-7[8H]-yl)-1-(2,4,5-trifluorophenyl)butan-2-amine; saxagliptin (BMS 477118, Bristol-Myers Squibb) (1S,3S,5S)-2-[(2S)-2-amino-2-(3-hydroxyadamantan-1-yl)acetyl]-2-azabicyclo [3.1.0]hexane-3-carbonitrile; 2,2,2-trifluoroacetic acid; alogliptin (Takeda) 2-({6-[(3R)-3-aminopiperidin-1-yl]-3-methyl-2,4-dioxo-1,2,3,4-tetrahydropyrimidin-1-yl}methyl)benzonitrile; NN701 (Novo-Nordisk); ABT-279 (Abbott) 2-(4-(2-((2 S,5R)-2-cyano-5-ethynylpyrrolidin-1-yl)-2-oxoethylamino)-4-methylpiperidin-1-yl)isonicotinic acid (CA Registry No. 676559-83-4); BI 1356 (Boehringer Ingleheim); SK-0403 (Sanwa Kagaku Kenkyusho); ALS 2-0426 (Amgen/Alantos) (S)-1-((S)-2-amino-3-((1S,4S)-5-(3-fluorophenylsulfonyl)-3-oxo-2,5-diazabicyclo[2.2.1]heptan-2-yl)propanoyl)pyrrolidine-2-carbonitrile; and PT630 (Point Therapeutics) 4-amino-5-((R)-2-boronopyrrolidin-1-yl)-5-oxopentanoic acid. The dose for DPPIV inhibitors is typically from 1 to 400 milligrams once or twice per day, preferably from 25 to 100 milligrams once or twice per day. In clinical trials of vildagliptin the doses ranged from 25 milligrams once daily to 100 milligrams twice daily. The usual dose of sitagliptin ranges from 25 mg once daily to 100 mg once daily depending on whether the patient has renal insufficiency and the degree of renal insufficiency.

### Use in Methods of Treatment

This invention provides a method for treating a mammalian subject with a condition selected from the group consisting of insulin resistance syndrome, diabetes (both Type I Diabetes and Type II Diabetes), secondary nonessential diabetes, and polycystic ovary syndrome, comprising administering to the subject a compound of Formula I or pharmaceutically acceptable salt thereof and a compound selected from an incretin mimetic and a DPPIV inhibitor in a combined amount effective to treat the condition. In accordance with the method of this invention a symptom of diabetes or the chance of developing a symptom of diabetes, such as atherosclerosis, obesity, hypertension, hyperlipidemia, fatty liver disease, nephropathy, neuropathy, retinopathy, foot ulceration and cataracts, each such symptom being associated with diabetes, can be reduced. This invention also provides a method for treating hyperlipidemia comprising administering to the subject an amount of a biologically active agent as described herein effective to treat the condition. Compounds reduce serum triglycerides and free fatty acids in hyperlipidemic animals. This invention also provides a method for treating cachexia comprising administering to the subject an amount of a biologically active agent as described herein effective to treat the cachexia. This invention also provides a method for treating obesity comprising administering to the subject an amount of a biologically active agent as described herein effective to treat the condition. This invention also provides a method for treating a condition selected from atherosclerosis or arteriosclerosis comprising administering to the subject an amount of a biologically active agent as described herein effective to treat the condition. The active agents of this invention are effective to treat hyperlipidemia, fatty liver disease, cachexia, obesity, atherosclerosis or arteriosclerosis whether or not the subject has diabetes or insulin resistance syndrome. The Compound of Formula I or salt thereof and the incretin mimetic or DPPIV inhibitor can be administered by any conventional route of systemic administration. Preferably the Compound of Formula I is administered orally. Other routes of administration that can be used in accordance with this invention include rectally, parenterally, by injection (e.g. intravenous, subcutaneous, intramuscular or intraperitioneal injection), or nasally. Exendins are preferably administered by injection, most preferably by subcutaneous injection.

Further embodiments of each of the uses and methods of treatment of this invention comprise administering any of the embodiments of the Compound of Formula I or pharmaceutically salts thereof and any of the incretin mimetics or DPPIV inhibitors described above. In the interest of avoiding unnecessary redundancy, each such agent and group of agents is not being repeated, but they are incorporated into this description of uses and methods of treatment as if they were repeated.

Many of the diseases or disorders that are addressed by this invention fall into two broad categories: Insulin resistance syndromes and consequences of chronic hyperglycemia. Dysregulation of fuel metabolism, especially insulin resistance, which can occur in the absence of diabetes (persistent hyperglycemia) per se, is associated with a variety of symptoms, including hyperlipidemia, atherosclerosis, obesity, essential hypertension, fatty liver disease (NASH; nonalcoholic steatohepatitis), and, especially in the context of cancer or systemic inflammatory disease, cachexia. Cachexia can also occur in the context of Type I Diabetes or late-stage Type II Diabetes. By improving tissue fuel metabolism, active agents of the invention are useful for preventing or amelioriating diseases and symptoms associated with insulin resistance. While a cluster of signs and symptoms associated with insulin resistance may coexist in an individual patient, it many cases only one symptom may dominate, due to individual differences in vulnerability of the many physiological systems affected by insulin resistance. Nonetheless, since insulin resistance is a major contributor to many disease conditions, drugs which address this cellular and molecular defect are useful for prevention or amelioration of virtually any symptom in any organ system that may be due to, or exacerbated by, insulin resistance.

When insulin resistance and concurrent inadequate insulin production by pancreatic islets are sufficiently severe, chronic hyperglycemia occurs, defining the onset of Type II diabetes mellitus (NIDDM). In addition to the metabolic disorders related to insulin resistance indicated above, disease symptoms secondary to hyperglycemia also occur in patients with NIDDM. These include nephropathy, peripheral neuropathy, retinopathy, microvascular disease, ulceration of the extremities, and consequences of nonenzymatic glycosylation of proteins, e.g. damage to collagen and other connective tissues. Attenuation of hyperglycemia reduces the rate of onset and severity of these consequences of diabetes. Because active agents and compositions of the invention help to reduce hyperglycemia in diabetes, they are useful for prevention and amelioration of complications of chronic hyperglycemia. The agents and compositions of the invention are useful for preventing or slowing down the progression from pre-diabetes (insulin resistance and/or impaired glucose tolerance) to Type II diabetes.

Both human and non-human mammalian subjects can be treated in accordance with the treatment method of this invention. The optimal dose of a particular active agent of the invention for a particular subject can be determined in the clinical setting by a skilled clinician. In the case of oral administration to a human for treatment of disorders related to insulin resistance, diabetes, hyperlipidemia, fatty liver disease, cachexia or obesity the Compound of Formula I or pharmaceutically acceptable salt thereof is generally administered in a daily dose of from 1 mg to 400 mg, more preferably from 200 mg to 400 mg, administered once or twice per day. In the case of oral administration to a mouse Compound of Formula I or pharmaceutically acceptable salt thereof is generally administered in a daily dose from 1 to 300 mg of the agent per kilogram of body weight. Incretin mimetics and DPPIV inhibitors are administered in accord with standard clinical practice. In some cases, coadministration with a compound of Formula I or a pharmaceutically acceptable salt thereof will improve the efficacy of other classes of drugs, permitting lower (and therefore less toxic) doses of such agents to be administered to patients with satisfactory therapeutic results. Exendin-4 is typically administered subcutaneous injection in a dose of either 5 micrograms of 10 micrograms twice-a-day.

In an embodiment of this invention the dose of the incretin mimetic or the DPPIV inhibitor is less than the therapeutic dose when the drugs are used alone. Typically the dose can be reduced to between 25% and 75% of the usual dose. In an embodiment of this invention the dose of either or both of the Compound of Formula I and the incretin mimetic or DPPIV inhibitor are chosen so that weight loss and/or appetite reduction result.

When the active ingredients are not mixed together to form a single admixture or composition they can be provided in the form of a kit comprising one or more unit oral doses of a Compound of Formula I or a pharmaceutically acceptable salt thereof, one or more unit doses of a an incretin mimetic or DPPIV inhibitor, and instructions for administering them in combination. Preferably the components of the kit are packaged together, such as in a box or a blister pack.

Type I Diabetes Mellitus: A patient with Type I diabetes manages their disease primarily by self-administration of one to several doses of insulin per day, with frequent monitoring blood glucose to permit appropriate adjustment of the dose and timing of insulin administration. Chronic hyperglycemia leads to complications such as nephropathy, neuropathy, retinopathy, foot ulceration, and early mortality; hypoglycemia due to excessive insulin dosing can cause cognitive dysfunction or unconsciousness. A patient with Type I diabetes is treated with an incretin mimetic or a DPPIV inhibitor and from 1 to 400 mg/day of the compound of Formula I or salt thereof, each drug separately as a single or a divided daily dose in the case where the incretin mimetic or the DPPIV inhibitor is used in combination with the Compound of Formula I, or both drugs combined as a single or a divided daily dose in the case where the DPPIV inhibitor is used in combination with the Compound of Formula I. The anticipated effect will be a reduction in the dose or frequency of administration of insulin required to maintain blood glucose in a satisfactory range, and a reduced incidence and severity of hypoglycemic episodes. Clinical outcome is monitored by measurement of blood glucose and glycosylated hemoglobin (an index of adequacy of glycemic control integrated over a period of several months), as well as by reduced incidence and severity of typical complications of diabetes. The treatment of this invention can be administered in conjunction with islet transplantation to help maintain the anti-diabetic efficacy of the islet transplant. Although exenatide alone is not recommended for Type I diabetes, the pancreas protection afforded by the Compound of Formula I makes the combination useful in treating Type I diabetes.

Type II Diabetes Mellitus: A typical patient with Type II diabetes (NIDDM) manages their disease by programs of diet and exercise as well as by taking medications such as metformin, glyburide, repaglinide, rosiglitazone, or acarbose, all of which provide some improvement in glycemic control in some patients, but none of which are free of side effects or eventual treatment failure due to disease progression. Islet failure occurs over time in patients with NIDDM, necessitating insulin injections in a large fraction of patients. It is anticipated that daily treatment in accordance with this invention (with or without additional classes of antidiabetic medication) will improve glycemic control, reduce the rate of islet failure, and reduce the incidence and severity of typical symptoms of diabetes. In addition, elevated serum triglycerides and fatty acids will be reduced, thereby reducing the risk of cardiovascular disease, a major cause of death of diabetic patients. As is the case for all other therapeutic agents for diabetes, dose optimization is done in individual patients according to need, clinical effect, and susceptibility to side effects.

GLP-1 analogs and modulators are more effective when islet mass and function is not substantially reduced, e.g. in early stage Type II diabetes. In later stage diabetes in the setting of islet failure, wherein patients become dependent on exogenous insulin, these agents are still active, but less so than at the earlier stage. From previous studies, it was known that Compound BI preserves islet insulin content in db/db mice, a model of diabetes featuring both insulin resistance and islet failure.

Hyperlipidemia: Elevated triglyceride and free fatty acid levels in blood affect a substantial fraction of the population and are an important risk factor for atherosclerosis and myocardial infarction. Treatment in accordance with this invention is useful for reducing circulating triglycerides and free fatty acids in hyperlipidemic patients. Hyperlipidemic patients often also have elevated blood cholesterol levels, which also increase the risk of cardiovascular disease. Cholesterol-lowering drugs such as HMG-CoA reductase inhibitors (“statins”) can be administered to hyperlipidemic patients in addition to agents of the invention, optionally incorporated into the same pharmaceutical composition.

Fatty Liver Disease: A substantial fraction of the population is affected by fatty liver disease, also known as nonalcoholic steatohepatitis (NASH); NASH is often associated with obesity and diabetes. Hepatic steatosis, the presence of droplets of triglycerides with hepatocytes, predisposes the liver to chronic inflammation (detected in biopsy samples as infiltration of inflammatory leukocytes), which can lead to fibrosis and cirrhosis. Fatty liver disease is generally detected by observation of elevated serum levels of liver-specific enzymes such as the transaminases ALT and AST, which serve as indices of hepatocyte injury, as well as by presentation of symptoms which include fatigue and pain in the region of the liver, though definitive diagnosis often requires a biopsy. The anticipated benefit is a reduction in liver inflammation and fat content, resulting in attenuation, halting, or reversal of the progression of NASH toward fibrosis and cirrhosis.

### Pharmaceutical Compositions

This invention provides a pharmaceutical composition comprising a compound of Formula I and a pharmaceutically acceptable carrier. Further embodiments of the pharmaceutical composition of this invention comprise any one of the embodiments of the biologically active agents described above. In the interest of avoiding unnecessary redundancy, each such agent and group of agents is not being repeated, but they are incorporated into this description of pharmaceutical compositions as if they were repeated.

Preferably the composition is adapted for oral administration, e.g. in the form of a tablet, coated tablet, dragee, hard or soft gelatin capsule, solution, emulsion or suspension. In general the oral composition will comprise from 1 mg to 400 mg, preferably from 200 mg to 400 mg, of the compound of Formula I or its salt. It is convenient for the subject to swallow one or two tablets, coated tablets, dragees, or gelatin capsules per day. However the composition can also be adapted for administration by any other conventional means of systemic administration including rectally, e.g. in the form of suppositories, parenterally, e.g. in the form of injection solutions, or nasally.

The active ingredients can be processed with pharmaceutically inert, inorganic or organic carriers for the production of pharmaceutical compositions. Lactose, corn starch or derivatives thereof, talc, stearic acid or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active ingredient no carriers are, however, usually required in the case of soft gelatin capsules, other than the soft gelatin itself. Suitable carriers for the production of solutions and syrups are, for example, water, polyols, glycerol, vegetable oils and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, coating agents or antioxidants. They can also contain still other therapeutically valuable substances, particularly antidiabetic or hypolipidemic agents that act through other mechanisms.

Exendin-4 amide (exenatide) is marketed by Amylin Pharmaceuticals, Inc. and Eli Lilly and Company under the tradename BYETTA in a pharmaceutical formulation containing exendin-4 amide, metacresol, mannitol, glacial acetic acid, sodium acetate trihydrate, and water.

This invention also provides a pharmaceutical composition comprising a compound of Formula I, a DPPIV inhibitor and a pharmaceutically acceptable carrier. DPPIV inhibitors are orally active and could therefore be combined in the same formulation as compounds of Formula I, or alternatively both drugs could be administered in separate tablets or capsules.

The invention will be better understood by reference to the following examples which illustrate but do not limit the invention described herein.

### EXAMPLES

### Example 1

In late-stage db/db mice, Compound BI was compared with Exendin-4 amide (obtained from Bachem) for efficacy in lowering blood glucose and preserving islet insulin content. Furthermore, a combination of the two drugs was tested.

### Groups

Vehicle
Compound BI (100 mg/kg/day p.o.)
Exendin-4 amide (10 microgram/day i.p.)
   10 microgram Exendin-4 amide i.p. per day attenuated hyperglycemia for up to 12 weeks when initiated in 4½ week old db/db mice (Greig et al., 1999 Diabetologia 42:45-50)
Compound BI (100 mg/kg/day+Exendin-4 amide 10 microgram/day i.p.)

Mice were treated daily for 4 weeks. Serum glucose and insulin and pancreatic insulin were measured.

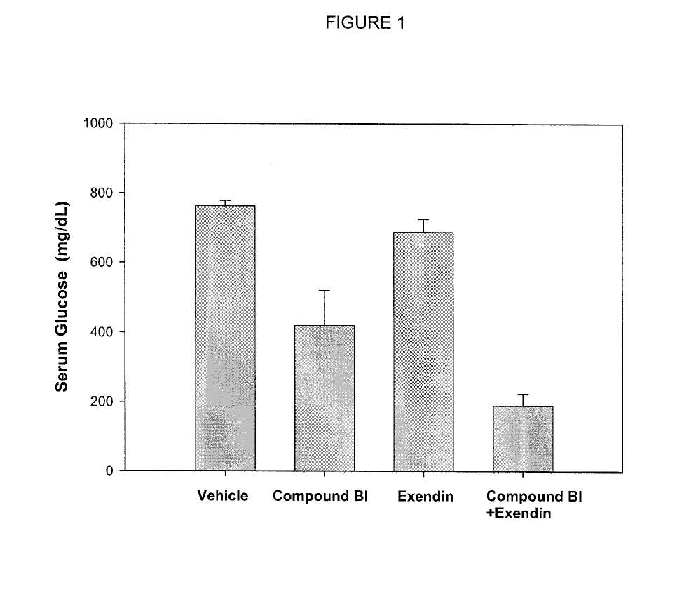

Vehicle-treated db/db mice were severely hyperglycemic after 4 weeks of treatment. Compound BI reduced serum glucose substantially, but Exendin-4 amide alone had little effect. The combination of Compound BI and Exendin-4 amide, however, had a strong effect on serum blood glucose. (FIG. 1).

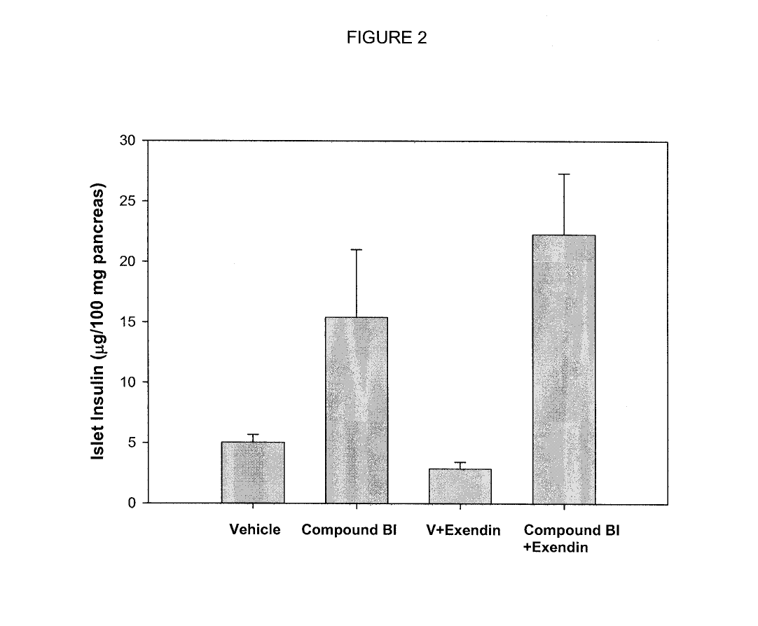

Islet insulin was low in animals treated with either Vehicle or Exendin-4 amide alone. Mice treated with Compound BI had more insulin than either of those two groups, and mice treated with Compound BI plus Exendin-4 amide had still more insulin in their islets. This is consistent with the idea that Exendin-4 amide acts on islets and that Compound BI enabled greater activity of Exendin-4 amide by preserving islets sufficiently for the Exendin-4 amide to have a functional target for its pharmacological activity. (FIG. 2)

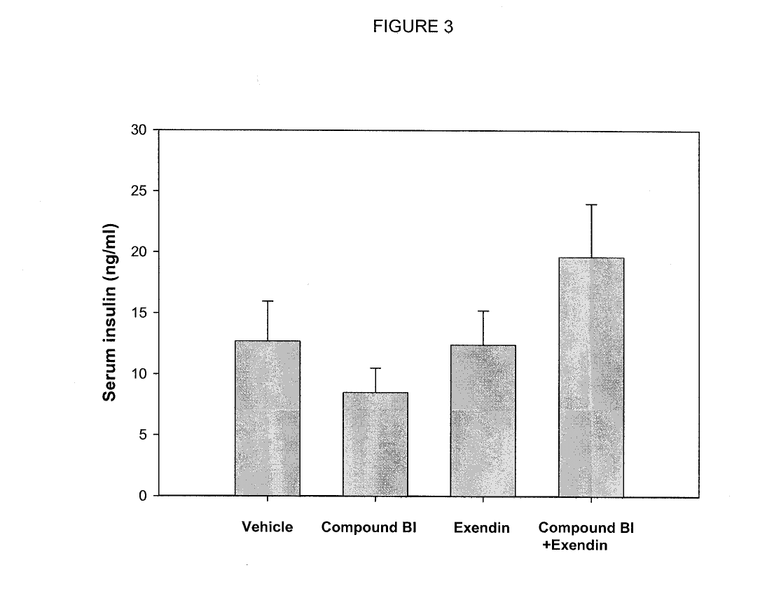

Serum insulin was also measured. The glucose-lowering achieved with Compound BI versus vehicle or Exendin-4 amide was attained without a significant increase in serum insulin, indicating that Compound BI reduced insulin resistance in this model. The slight elevation of serum insulin in mice treated with Compound BI and Exendin-4 amide is consistent with the known effect of Exendin-4 of causing insulin secretion. (FIG. 3)

### Example 2

Peripheral blood glucose levels of db/db mice was monitored. When the glucose levels exceeded 400 mg/dL the mice were divided into groups of 6 mice each. The mice in each group were given either vehicle or Compound BI (100 mg/kg by oral gavage)+/−i.p. injections of exendin-amide (Bachem, King of Prussia, Pa.) as indicated below for 4 weeks.
1) Vehicle
2) Vehicle+Exendin (3 μg/kg)
3) Vehicle+Exendin (10 μg/kg)
4) Compound BI
5) Compound BI+Exendin (3 μg/kg)
6) Compound BI+Exendin (10 μg/kg)

After four weeks of treatment, the mice were bled via the retroorbital sinus and sera sent to Anilytics, Inc. (Gaithersburg, Md.) for analysis of circulating glucose levels. The pancreata were collected, weighed, flash frozen and then processed for pancreatic insulin assays. Briefly, the pancreas was placed into pancreatic extraction solution (75% ethanol and 25% 0.15N HCl) and the volume adjusted to 1 mL per 100 mg of pancreas. The pancreas was then sonicated and stored at −20° C. overnight. The next day, the samples were centrifuged at 2500 rpm for 5 minutes at 4° C. to pellet unsolubilized material. 1.5 mL of supernatant was placed in an Eppendorf tube and spun in a microcentrifuge at 1300 rpm for 20 minutes at 4° C. to pellet any remaining unsolubilized material. The resulting supernatant was then assayed for the presence of insulin using an electrochemiluminescent (ECL) assay developed for mouse insulin. This assay uses rat insulin (Lincon Research Inc., St. Charles, Mo.) as a standard but shows linearity with mouse insulin as well. The assay uses a biotinylated anti-mouse insulin monoclonal antibody (Clone 5E4/3 from Biogenesis, a division of AbDSerotec, Raleigh, N.C.) as the capture antibody and a ruthenium labeled anti-mouse insulin antibody (Clone 5B6/6 from Biogenesis). The antibodies are directed against different epitopes on the insulin molecule and were labeled according to the direction of the manufacturer of the ECL assay reagents (BioVeris Corp., Gaithersburg, Md.). Standards or samples were mixed with the two antibodies and incubated for 2 hours at room temperature with shaking. Subsequently, Dynabeads coated with streptavidin (BioVeris Corp., Gaithersburg, Md.) were added and incubated for an additional 30 minutes at room temperature with shaking. The samples were then read on an M384 instrument (BioVeris Corp., Gaithersburg, Md.). The amount of insulin in the sample was proportional to the amount of light emitted.

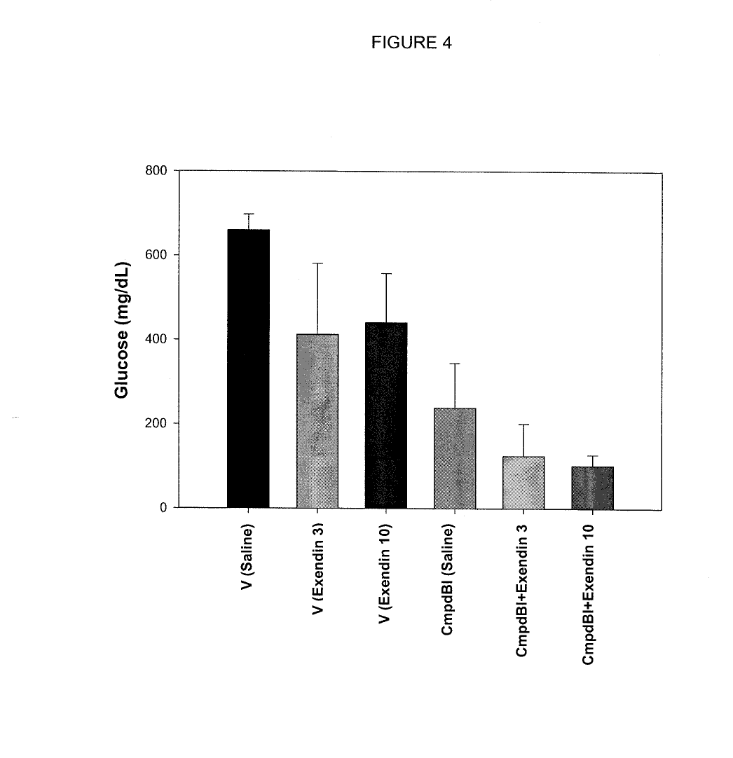

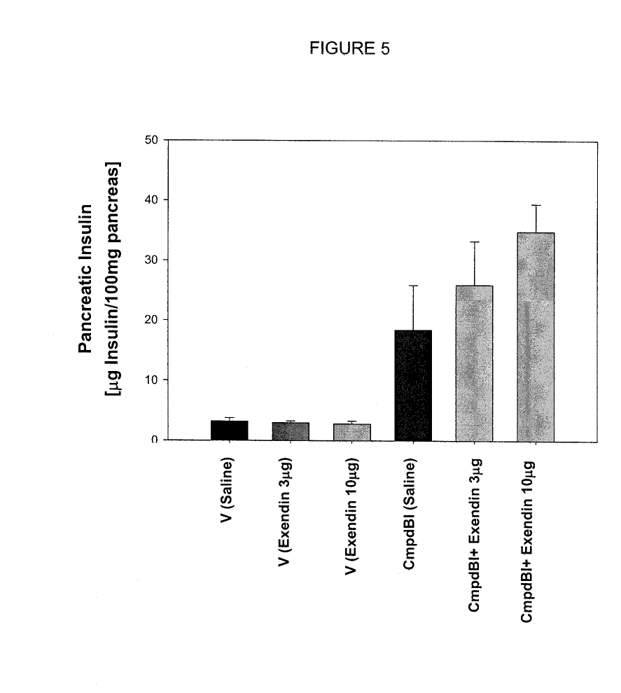

FIG. 4 shows the effects of Compound BI, Exendin or the combination of the two drugs on circulating glucose levels. Normal mouse circulating glucose levels are 124-262 mg/dL (Anilytics, Inc. Gaithersburg, Md.). Mice treated with saline alone exhibited high levels of hyperglycemia (FIG. 4) and low pancreatic insulin levels (FIG. 5). The low pancreatic insulin levels are due to the transition from insulin resistance in these mice to frank diabetes. Treatment with exendin at either 3 μg/kg or 10 μg/kg for 4 weeks showed a slight reduction in circulating glucose levels but no significant change in pancreatic insulin. Mice treated with Compound BI for four weeks showed a significant reduction in circulating glucose levels and a corresponding increase in pancreatic insulin (FIG. 5). Mice treated with the combination of Compound BI and exendin at 3 μg/kg or 10 μg/kg for four weeks showed a dose-responsive, synergistic reduction in circulating glucose levels and increase in pancreatic insulin levels.

### Example 3

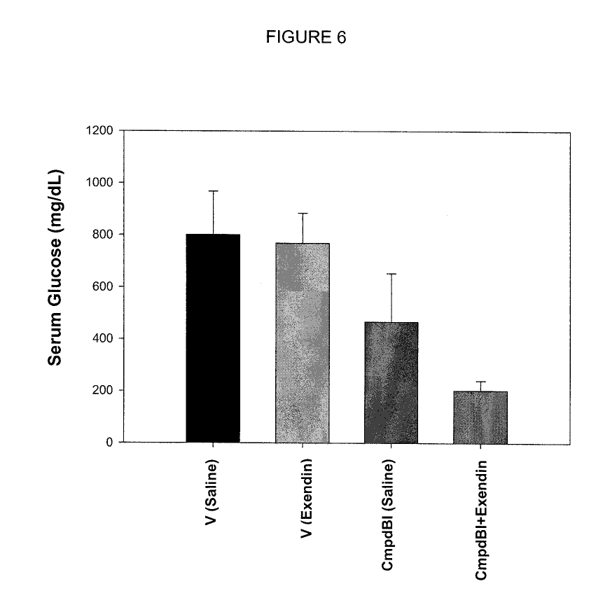

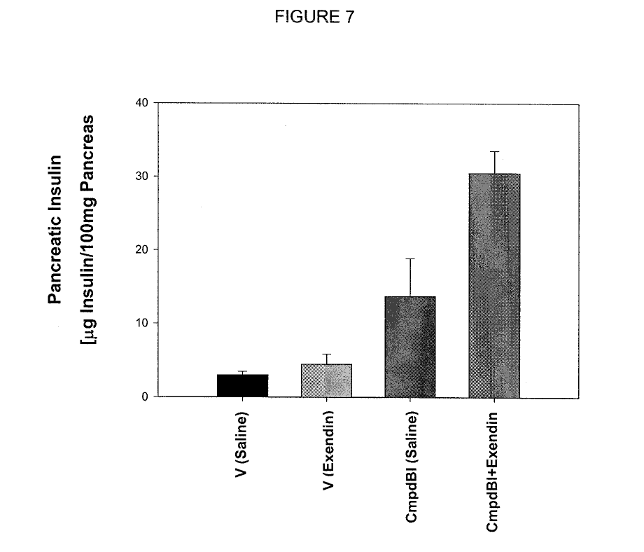

In a separate experiment, db/db mice were treated as in Example 2. Again, a synergistic effect of treatment with the combination of Compound BI (100 mg/kg p.o.) and exendin-amide (10 μg/kg, i.p.) was noted on both circulating glucose (FIG. 6) and on pancreatic insulin (FIG. 7).

### Example 4

A widely used model for Type 1 diabetes is Streptozotocin-treated mice. Streptozotocin (STZ) is an antibiotic produced by Streptomyces achromogenes. The structure of STZ is a glucose molecule with a highly reactive nitrosourea side chain; at an appropriate dosage, STZ is selectively toxic to pancreatic beta cells. This model was used to examine the effects of the combination of Compound BI plus exendin-amide on circulating glucose levels, circulating C-peptide levels (a measure of pancreatic insulin secretion into the blood stream) and total pancreatic insulin.

Food was removed from C57B1/6J mice in the morning on Day 1 and the mice were given an injection of STZ (160 mg/kg) in the afternoon. Glucose was measured by tail bleed on Day 4 and mice with glucose readings higher than 350 mg/dL were divided into groups of 10 mice each. Treatments (Vehicle (saline), or a combination of Compound BI (100 mg/kg, p.o.) and exendin-amide (10 μg/kg, i.p.) were begun on the same day (i.e., Day 4). On Day 18, glucose was again measured by tail bleed. Mice were sacrificed on Day 30. Circulating glucose and pancreatic insulin was measured as in Example 2; serum insulin levels were measured using the same ECL assay as for pancreatic insulin. Circulating levels of C-peptide were measured in serum using a commercial ELISA (Babco, Richmond, Calif.) and compared to levels in normal (untreated) C57B1/6J mice.

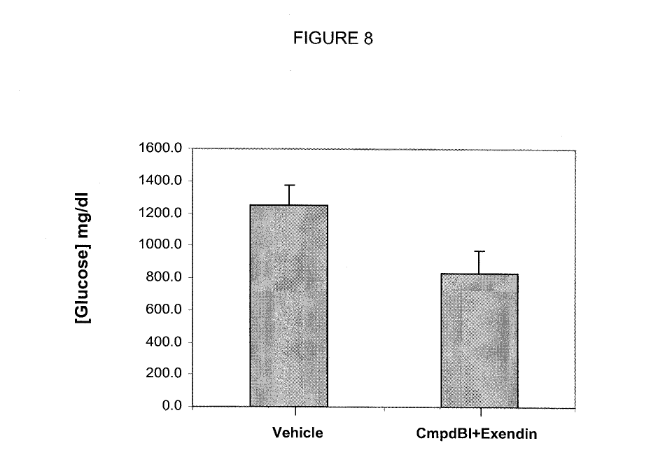

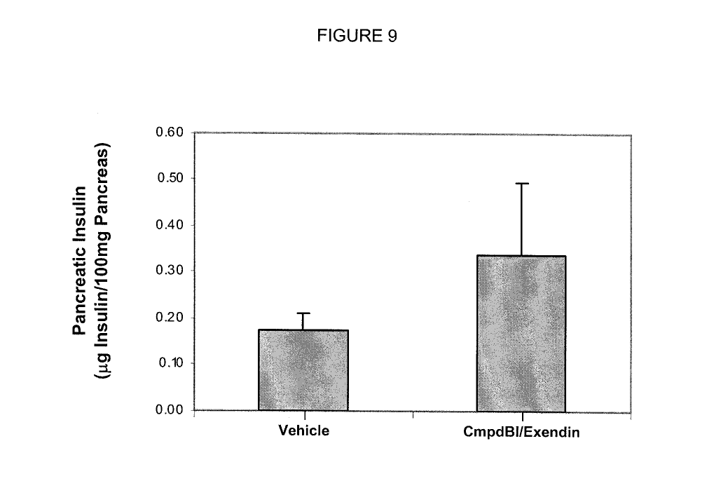

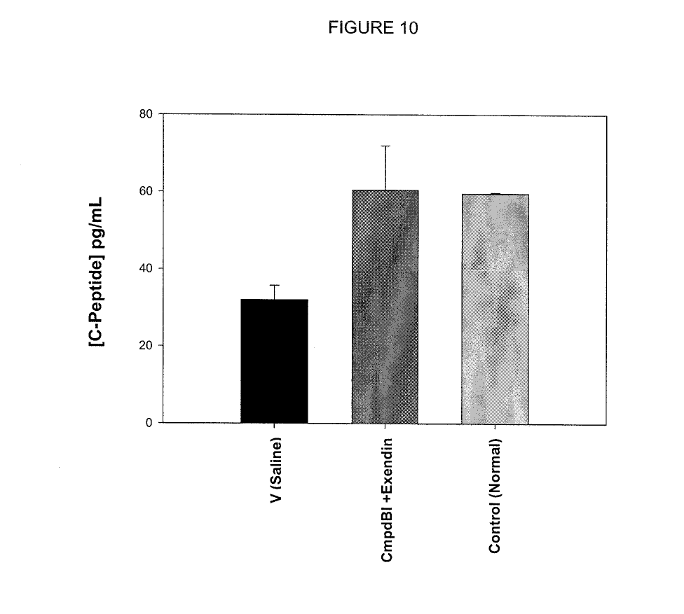

Mice treated with the combination of Compound BI and exendin-amide showed a reduction in circulating glucose levels (FIG. 8) and an increase in circulating C-peptide (to normal levels, FIG. 9) as well as pancreatic insulin (FIG. 10).

### Example 5

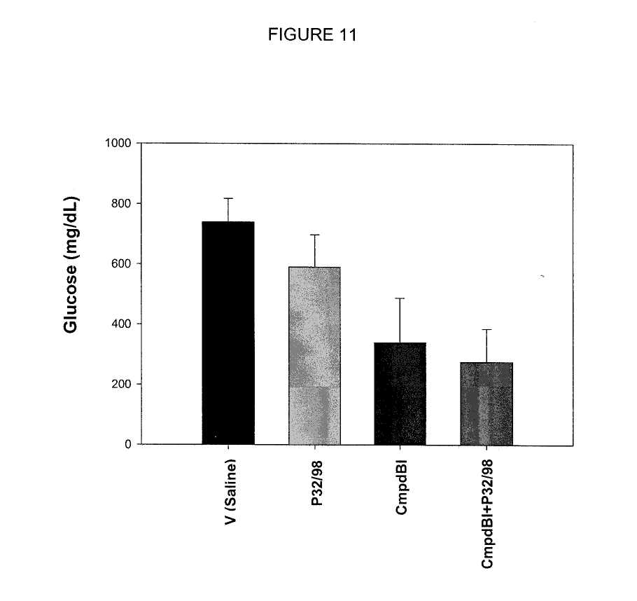

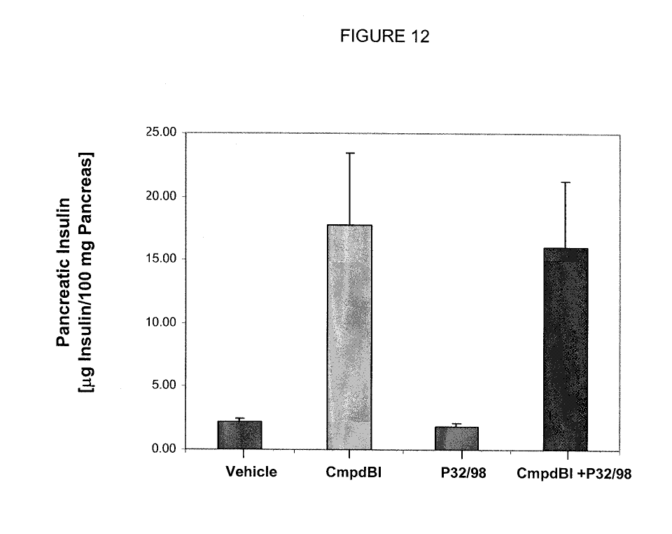

P32/98 is a DPPIV inhibitor that has been shown to be active in streptozotocin-induced diabetes (Pospisilik et al., Diabetes 52:741-750, 2003). Ten week-old db/db mice were given vehicle or Compound BI (100 mg/kg, p.o.)+/−P32/98 (10 mg/kg, twice a day by gavage) as indicated for 4 weeks. Assays for circulating glucose and pancreatic insulin were conducted as in Example 3. P32/98 treated mice showed a slight reduction in circulating glucose levels but no increase in pancreatic insulin. Compound BI also reduced circulating glucose levels and showed a further reduction when combined with P32/98 (FIG. 11). Compound BI increased pancreatic insulin levels when used by itself; there was no further increase in pancreatic insulin when combined with P32/98 under these conditions, most likely because of the strong efficacy seen with Compound BI as a monotherapy under these conditions (FIG. 12).

1. A method of treating a mammalian subject having a condition selected from the group consisting of insulin resistance syndrome, diabetes, polycystic ovary syndrome, hyperlipidemia, fatty liver disease, cachexia, obesity, atherosclerosis and arteriosclerosis, comprising administering to the subject a Compound of Formula I or a pharmaceutically acceptable salt thereof 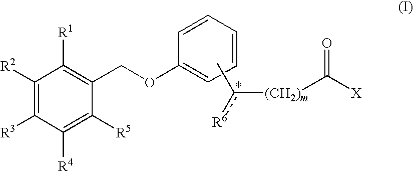
wherein:
m is 0, 2 or 4; and
X is —OR⁷, wherein R⁷ is hydrogen or alkyl having from 1 to 3 carbon atoms;
R⁶ is hydrogen, O or hydroxy; and
three of R¹, R², R³, R⁴ and R⁵ are hydrogen and the remainder are independently selected from the group consisting of hydrogen, halo, hydroxy, methyl, ethyl, perfluoromethyl, methoxy, ethoxy, and perfluoromethoxy;
or X is —NR⁸R⁹, wherein R⁸ is hydrogen or hydroxy and R⁹ is hydrogen, methyl or ethyl;
R⁶ is hydrogen; and
three of R¹, R², R³, R⁴ and R⁵ are hydrogen and the remainder are independently selected from the group consisting of hydrogen, halo, methyl, ethyl, perfluoromethyl, methoxy, ethoxy, and perfluoromethoxy;
in combination with an incretin mimetic or a dipeptidyl peptidase IV inhibitor in a combined amount effective to treat the metabolic condition.

2. The method of claim 1, wherein R¹ is methyl and R⁵ is methyl.

3. The method of claim 1, wherein X is —OR⁷, wherein R⁷ is hydrogen or alkyl having from 1 to 3 carbon atoms.

4. The method of claim 1, wherein X is —NR⁸R⁹, wherein R⁸ is hydrogen or hydroxy and R⁹ is hydrogen, methyl or ethyl.

5. The method of claim 1, wherein the Compound is represented by Formula IA.

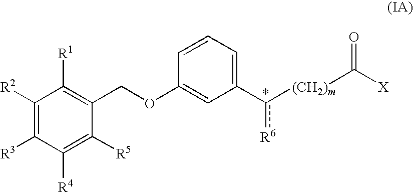

6. The method of claim 5, wherein R¹ is methyl and R⁵ is methyl.

7. The method of claim 6, wherein the Compound is 4-(3-(2,6-Dimethylbenzyloxy)phenyl)-4-oxobutyric acid.

8. The method of claim 6, wherein the Compound is 3-(2,6-Dimethylbenzyloxy)-phenylacetic acid.

9. The method of claim 6, wherein the Compound is 4-3-(2,6-Dimethylbenzyloxy)-phenyl)-4(R)-hydroxybutanoic acid.

10. The method of claim 6, wherein the Compound is N-Hydroxy-2-[3-(2,6-dimethylbenzyloxy)phenyl]acetamide.

11. The method of claim 1, wherein the incretin mimetic is selected from the group consisting of an exendin, an exendin agonist, a non-peptide small molecule GLP-1 receptor agonist, their polymer-modified and acylated forms and pharmaceutically acceptable salts, hydrates, and solvates, and hydrates and solvates of such salts.

12. The method of claim 1, wherein the exendin is exendin-4 or exendin-4 amide.

13. The method of claim 1, wherein the dipeptidyl peptidase IV inhibitor is selected from the group consisting of vildagliptin, sitagliptin, saxagliptin, alogliptin, ABT-279, BI 1356, ALS 2-0426 and PT630.

14. The method of claim 1, wherein the subject is a human.

15. The method of claim 1, wherein the incretin mimetic or the dipeptidyl peptidase IV inhibitor is administered in an amount that is less than the usual therapeutic dose when administered alone.

16. The method of claim 1, wherein the combined amount is selected so that the treatment results in one or more of weight loss and appetite reduction in the subject.

17. The method of claim 1, wherein the Compound of Formula I is administered orally and the incretin mimetic is administered by subcutaneous injection.

18. The method of claim 1, wherein the condition is pre-diabetes or Type II diabetes.

19. The method of claim 18, wherein the pre-diabetes comprises one or both of insulin resistance and impaired glucose tolerance.

20. The method of claim 1, wherein the treatment reduces a symptom of Type II diabetes or the chances of developing a symptom of Type II diabetes, wherein the symptom is selected from the group consisting of: atherosclerosis, obesity, hypertension, hyperlipidemia, fatty liver disease, nephropathy, neuropathy, retinopathy, foot ulceration and cataracts, associated with Type II diabetes.

21-38. (canceled)
